(19) United States
(12) Patent Application Publication
(10) Pub. No.: US 2007/0027388 A1
(43) Pub. Date: Feb. 1, 2007

(54) Patch-type physiological monitoring apparatus, system and network

(76) Inventor: Chang-An Chou, Taipei (TW)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 11/494,881

(22) Filed: Jul. 28, 2006

(30) Foreign Application Priority Data
Aug. 1, 2005 (CN) 200510084597.3
Mar. 14, 2006 (CN) 200610067814.2

(51) Int. Cl. A61B 5/04

(52) U.S. Cl. 600/393; 600/372; 600/382; 600/509; 600/546; 600/544

(57) ABSTRACT

Patch-type physiological monitoring apparatus, system and network are disclosed. The patch-type physiological monitoring apparatus includes at least a node, and at least a patch for attaching to a skin surface of a user and for supporting the node on the skin surface through joining therewith, wherein the node includes at least a signal I/O port for externally connecting to at least a sensor or electrode through a connecting wire so as to acquire a physiological signal, and a RF module for transmitting and receiving signal. The apparatus according to the present invention is of light weight and compact size and easily attached to human body through adhesive patches. Through a RF module, the system can wirelessly communicate with corresponding devices without additional wiring. Further, the system can utilize conventional electrodes, patch electrodes and electrode wiring to avoid extra cost for facilities' renewal and replacement.

### TECHNICAL FIELD OF THE INVENTION

The present invention relates to a physiological monitoring system, and more particularly, to wireless, miniature and portable techniques employed in long-term physiological monitoring, hereby, reducing load, while retaining free mobility, of individuals under healthcare.

### BACKGROUND OF THE INVENTION

It has been realized that efficient utilization of available resource for public healthcare are hampered by upsurge demands from metropolitan residence, whose hasty living tempo sacrifices their time squeezed for medical attention, and also the fact of aging society in most developed and developing countries, draining out more and more resource in healthcare, whence comes a potential booming market of services in both homecare and point-of-care. The portable type of physiological monitoring system is thus of great interests and getting much preferred by users in diverse fields due to its less space restriction and easy operation. Unlike conventional clumsy full-function systems used in hospitals to monitor patients' physiological status, the portable system requires more than professional equipments, it is also an outfit with light weight, compact size, and convenient attachment to ease patients freely on the move under medical monitoring. All these features meet not just the needs on least space occupied by monitoring systems for most hospitals and clinics, but also satisfy users without much professional experience in various fields of healthcare, where handy operation and free mobility of patients are essential.

The adoption of wireless and portable techniques into physiological monitoring systems, actually, rendered the development of various handheld monitoring devices, and indeed, removed several deficiencies in conventional equipments. However, weight and size thereof are still issues to be concerned by users as applying these monitoring systems to different clinic conditions. For example, U.S. Pat. No. 6,611,705 disclosed a type of electrode connector for electrocardiogram (ECG) and its associated monitoring system, wherein the wireless electrode connectors are used to eliminate wire interference from wire connections between tester and equipment in traditional ECG measurement; in other word, the utilization of wireless techniques in the ECG monitoring is intended to improve signal quality by way of reducing wire interference, while the techniques and designs disclosed in that invention did not further contribute to lower burdens on testers and enhance ease of operation in field owing to size and weight still not dramatically abated. The testers are remained bound to monitoring equipments and hardly move at will, therefore, leaving much room for prior arts to improve.

On the other hand, as described in U.S. Pat. No. 6,368,287, a sleep apnea screening system was disclosed and embodied in the manner of compact size and flexible attachment such that testers can easily carry the device in test. However, the invention actually served as an event counting recorder and failed to report complete physiological status such as to either monitor sleep in real time, or analyze detailed sleep physiology after test. The design thereof is primarily aimed to fulfill the specific demand on pre-screening potential patients with sleep disorders because the fact of limited resource available for sleep test keeps a long waiting list in every sleep center. Such very idea was also applied to U.S. Pat. No. 6,597,944, wherein the attachment of device and display of counting results, again, represented the prior concept about pre-screening. Apparently, these two inventions as described above, though reach the purposes of compact size and free mobility but not definitely light weight, scarify detailed physiological information retrieving from testers, thus not adaptable to meet various requirements from different biomedical monitoring conditions, and thereby limit their application spectrum in clinic.

Accordingly, there indeed exists needs for physiological monitoring apparatus and system that are of easy operation and convenient for testers' movement, and, moreover, possesses complete recording ability in parallel to measuring physiological status. Apart from that, in consideration of prevailing conventional monitoring systems already broadly in use, it is critical to develop physiological monitoring device and system that fulfill the needs described above, while compatible to adopting existed equipments for cost reduction.

Therefore, the object of the present invention is to provide a physiological monitoring system that is of light weight and compact size and easily attached to human body through adhesive patches such that the demands on portable detection, as well as light weight and compact size are achieved.

Another object of the present invention is to further provide a physiological monitoring system, wherein through a RF module, the system can wirelessly communicate with corresponding devices without additional wiring, which may also cause signal interference.

Another advanced object of the present invention is to further provide a physiological monitoring system that can utilize conventional electrodes, patch electrodes and electrode wiring in the physiological monitoring to avoid extra cost for facilities' renewal and replacement.

### SUMMARY OF THE INVENTION

The present invention provides a physiological monitoring apparatus includes at least a node and at least a patch for attaching to a skin surface of a user and for supporting the node on the skin surface through joining therewith, wherein the node includes at least a signal I/O port for externally connecting to at least a sensor or electrode through a connecting wire so as to acquire a physiological signal, and a RF module for transmitting and receiving signal and/or a connecting port used for communication and power transmission with an external device.

A physiological monitoring network includes at least a physiological monitoring apparatus, at least a wireless device having a connecting port for communication and power transmission with the connecting port of the node, and a server system for real time monitoring, analyzing, processing, storing and/or informing related personnel, wherein the physiological monitoring apparatus includes at least a node having a connecting port used for communication and power transmission and a wireless transmission module, and at least a patch for attaching to a skin surface of a user and for supporting the node on the skin surface through joining therewith, and the communication between the physiological monitoring apparatus and the wireless device includes a handshaking and a communication between the wireless device and the server system is achieved in a wired or wireless manner.

### BRIEF DESCRIPTION OF THE DRAWINGS

A more detailed understanding of the invention may be had from the following description of a preferred embodiment, given by way of example, and to be understood in conjunction with the accompanying drawings, wherein:

FIG. 1 is a schematic view showing the circuitry arrangement of the physiological monitoring apparatus in the present invention;

FIGS. 2A˜2D are schematic views showing the methods for integrating the node and the adhesive patch of the physiological monitoring apparatus in the present invention;

FIG. 3 is a schematic view showing the node of the physiological monitoring apparatus in the present invention;

FIG. 4 is a schematic view showing the physiological monitoring apparatus having two nodes in the present invention;

FIG. 5 is an example illustrating the method for configuring distributive nodes in a 12-lead ECG measurement according to the present invention;

FIG. 6 is another example illustrating the method for configuring distributive nodes in a 12-lead ECG measurement according to the present invention;

FIGS. 7A˜7B show the exemplary connection methods between the node and external device in the present invention;

FIGS. 8A˜8B are schematic views showing the circuitry distributions in distributive multiple nodes of the physiological monitoring apparatus according to the present invention;

FIG. 9 is an example illustrating a physiological monitoring system including the physiological monitoring apparatus and a RF transceiver according to the present invention;

FIG. 10 is an example illustrating another physiological monitoring system including the physiological monitoring apparatus and another RF transceiver according to the present invention;

FIG. 11 shows another example of the structure for the RF transceiver in the present invention;

FIG. 12 is an example illustrating another physiological monitoring system including the physiological monitoring apparatus and a portable wireless operation device according to the present invention;

FIG. 13 is a schematic view showing the application of the physiological monitoring system in FIG. 12;

FIG. 14 is an illustration as the portable wireless operation device is performed as a watch according to the present invention;

FIG. 15 is a schematic view showing a physiological monitoring network in the present invention;

FIG. 16 is an example illustrating a physiological monitoring apparatus performed to have distributive multiple nodes with a master node included therein according to the present invention;

FIGS. 17A˜17B are examples illustrating the node in the physiological monitoring apparatus using an attaching element for attaching to the skin surface according to the present invention.

### DETAILED DESCRIPTION OF THE PREFERRED EMBODIMENTS

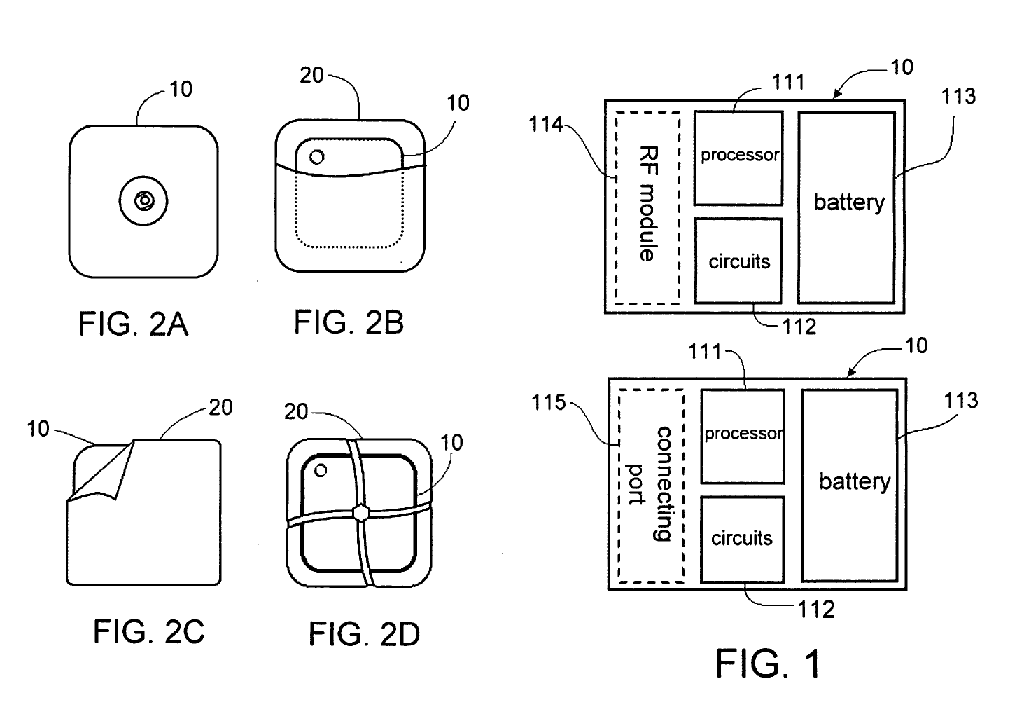

Please refer to FIGS. 1 to 3. According to the present invention, a physiological monitoring apparatus includes a node 10 joined with an adhesive patch 20 such as to render a sturdy support of the node 10 and also provide a strong attachment of the node 10 to user's skin surface through the adhesive patch 20. Herein, the node is defined as an integration with a housing capable of connecting with another node and/or connecting to external device.

A variety of join methods for integrating the adhesive patch 20 with the node 10 are illustrated in, but not limited to, FIG. 2, including snap fastener (FIG. 2A), pocket pouch (FIG. 2B), adhesive binder (FIG. 2C) and any type of join method that serves as a steady holder between node and adhesive patch and bolsters holding strength for node, such as strap fastener (FIG. 2D).

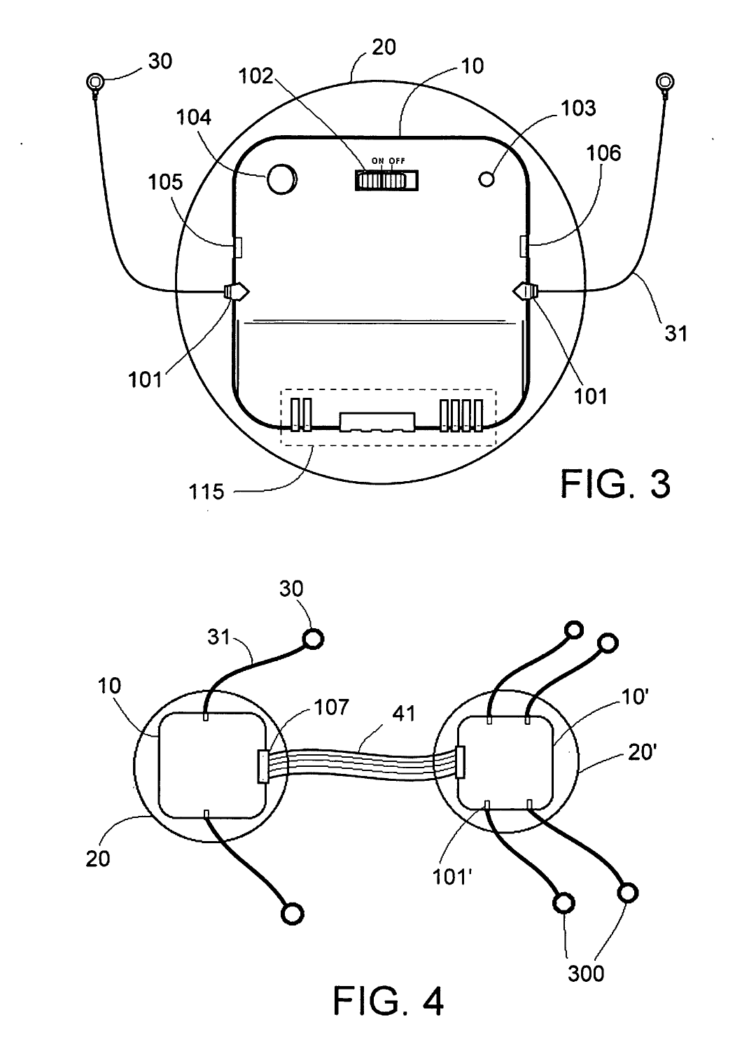

As illustrated in FIG. 3, the node 10 includes at least a signal I/O port 101 to connect to corresponding sensors (e.g. optical sensor of pulse oximeter) or electrodes 30 (e.g. ECG electrode) via a connecting wire 31 (e.g. the connecting wire used for the conventional electrode) to detect physiological signals. Accordingly, the number of sensor(s)/electrode(s) connected to the signal I/O port is not limited and can be pre-defined or preserved by designer based on what physiological parameters to be measured and what combinations of these electrodes/sensors to be grouped together. The connection between the signal I/O port and incoming wires, thereof, can be realized in terms of plug-in, connect-through-connector or direct-connection structure (as shown in FIG. 3).

An embodiment of the present invention is to apply patch-type physiological monitoring apparatus to detect cardiac bioelectricity. The placement of node and associated adhesive patch is appropriately allocated between electrode positions, for example, bilateral regions around heart area on front chest surface, to abide the principle of complexity reduction of electrode connections. Or, in the case of ⅗-lead system, the node and associated adhesive patch are attached to an appropriate position in the region within 5 electrode placement loci. Rather than other conventional ECG measurement that is simply a hand-held device or utilizes thorax/abdominal strap to hold their device, causing undesired inconvenience in monitoring process, the node in the present invention can be firmly rested on skin surface by associated patch due to its light weight and compact size. As the node all set, the monitoring apparatus is ready to go once the electrodes are attached to pre-determined loci and connected to corresponding interface on the node.

In a preferred embodiment, the node-associated adhesive patch per se can be also served as an electrode for bioelectrical detection, that is, one of five-lead electrodes or reference/ground electrode in the case of ⅗-lead system, such that maximal simplification of equipment set-up can be achieved. The implementation of such an adhesive electrode patch is exemplified by resting an electric conductive structure on an electrode patch, for example, the snap fastener electrode is namely a known electrode with an electric conductive snap fastener thereon, to connect electrode on the patch to corresponding signal I/O port on the node. Apart from being bioelectrical electrodes, the adhesive patch also can be a temperature sensor, such that a detachment of patch can be determined by detecting the abrupt variation in temperature, and of course, the temperature sensor in such a configuration also can be used to report user's body temperature.

As described above, the electric circuits in the node 10, as indicated in FIG. 1, include, but not limit to, a processor 111, functional circuitry 112, rechargeable battery set 113 and other circuits (not shown) that a skillful person in the field is familiar with, such as A/D converter and filters.

In addition, a switch 102 on the node is used to turn on/off the monitoring apparatus, and a display device 103 provided thereon demonstrates operation status, power level and system information. Furthermore, a push button 104 on the node 10 is designed to trigger a time marking to enable users to mark special events. For example, in one embodiment, as a user experiences unusual physical symptom during monitoring process, he or she can push the button 104 to mark the time for this special event, such that physician may analyze the recorded events and data to obtain correct diagnosis. Or, in another embodiment, as a user experiences unusual physical symptom, he or she can push the button 104 to start a recording in a defined interval, such as to assist physician to pinpoint physical abnormity of the user. Or, in another embodiment, the user can push the button 104 to ring for emergent help. To sum up, the function of the push button 104 can be implemented in various designs and quantity based on users' real demands.

Furthermore, a RF module 114 on the node 10 enables wireless communication with an external wireless device; while, on the other hand, a connecting port 115 for communication and power transmission can perform a data exchange, such as handshaking, with external devices, such as personal computers, via a wired connection.

Thereby, as the RF module is included in the node of the physiological monitoring apparatus, physiological signals are transmitted to external receiver through wireless communication. While, in case, only the connecting port is adopted in the node of the physiological monitoring apparatus, the physiological signals can be recorded and then transmitted to another device through the connecting port after the monitoring process is accomplished. In another embodiment that both the RF module and the connecting port are adopted in physiological monitoring, the RF module works mainly in the condition of the attaching period of the monitoring apparatus; and the connecting port, on the other hand, is used during the period of detachment of the monitoring apparatus. For example, according to the function of power transmission, the connecting port can be used to charging battery as the battery in used is rechargeable.

The above-described RF module can be realized as, but not limits to, Bluetooth, 802.11a, 802.11b, 802.11g, GPS, IrDA, and other RF module.

Moreover, the node may also entail a memory set (not shown) to render flexibility in system operations. Through memory allocation, the physiological monitoring apparatus in the present invention can store retrieved physiological signals in the memory set, and then, transmit saved data to another external device through above-described RF module or connecting port. By doing so, it will be much beneficial to power saving since real-time wireless transmission shall consume higher power usage. Such a concern becomes critical while battery is adopted as the power source.

In accordance with the above disclosure, the present invention even features an advantage that the quantity of node can be augmented and adjustable based on how many physiological parameters required to measure, without further increasing users' loading, due to node's compact size and light weight, as well as its attachment method. In an embodiment of one single monitoring with multiple retrieving points, such as 12-lead ECG and multi-channel electroencephalogram (EEG), multiple electrodes and associated circuitry may complicate system configuration and increase system's size and weight, so that, to reduce system complexity, it can be embodied in plural nodes associated with plural adhesive patches, which are also performed as the electrode patches used in the monitoring, for distributing the abundant circuitry as well as the increased volume and weight into plural interconnected nodes.

Accordingly, in a simplest embodiment of one single monitoring with multiple detecting points by employing plural nodes, FIG. 4 illustrates the condition of using two nodes in the physiological monitoring, where two distributive nodes 10 and 10′ are attached to skin surface through two associated adhesive electrode patches 20 and 20′ as described above. In such a configuration, for connecting the nodes 10 and 10′, an extension port 107 is provided on the node 10 for connecting to one end of a connecting wire 41, whose another end is connected to the node 10′. For the node 10′, a signal I/O port 101′ (the same as the signal I/O port 101 in FIG. 3) is provided thereon for externally connecting to additional electrodes and/or sensors 300. Thereby, without increasing node's size and user's loading, the physiological monitoring apparatus with multiple detecting points, such as 12-lead ECG, can be completed.

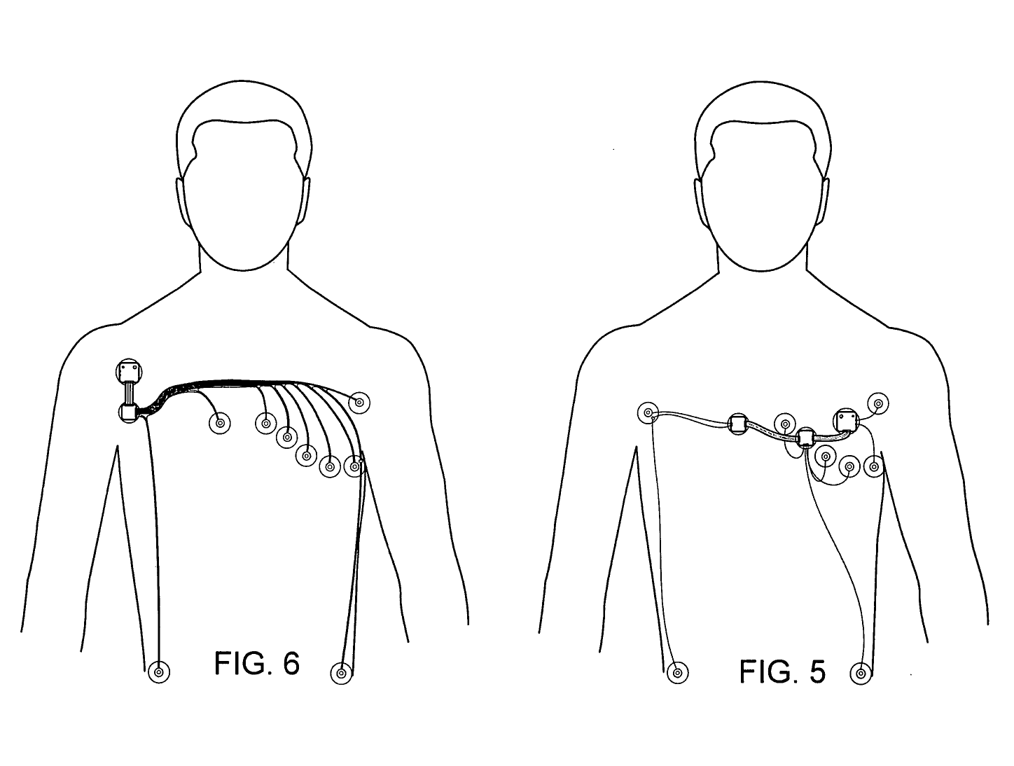

Besides, owing to the configuration of plural distributive nodes as well as the extension port and connecting wire, remote electrodes can be connected through intermediate nodes, as the case of 12-lead ECG shown in FIG. 5, for further simplifying wiring complexity and removing its potential tangling.

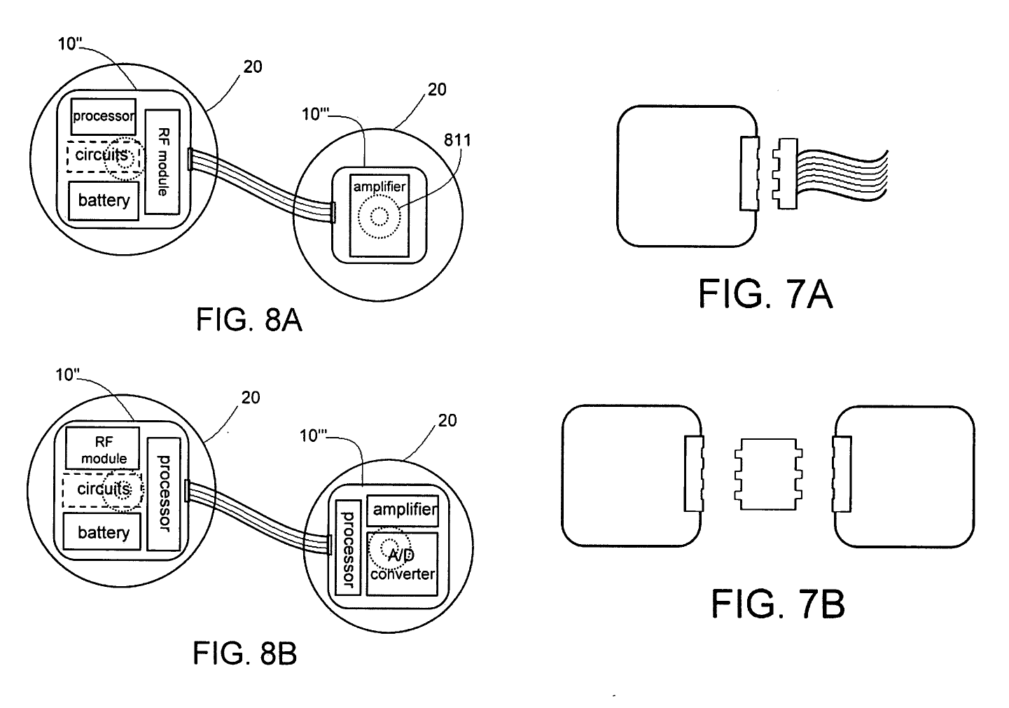

In particular, the interconnection between the extension port 107 and the associated connecting wire 41 can be achieved by plug-in, connect-through-connector (FIGS. 7A and 7B) or direct-connection (FIG. 4) structure. The join methods for respective node with associated adhesive patch may utilize snap fastener, pocket pouch, adhesive binder, strap fastener, and any type of join method that serves as a steady holder for the node.

In an advanced embodiment, the configuration of distributive nodes provide the possibility of alternative power supply if power input 105 and power output 106 are included in the node as shown in FIG. 3, by which one node can retrieve electric power from its neighboring node, and vice versa. Apparently, the configuration of distributive nodes disclosed in the present invention can further help the reduction of node's size since battery set occupies appreciated part of the node as indicated in FIG. 1; and, on the other hand, electric shortage during measurement can be avoid as neighboring node may also serve as a power supply if necessary.

It is noticed that the methods for configuring distributive nodes, including their locations and quantity, are versatile, and the design illustrated in FIG. 5 that utilizes conventional electrode cables for interconnections between the nodes employing electrode patches is mainly for the purpose of exemplification. It is likely to utilize other methods to adjust and simplify configuration of distributive nodes and electrodes. Flexible flat cable (FFC) and flexible printed circuit board (FPC) shown in FIG. 6 which provide good conducting and adhesive properties and avoid wire tangling are another examples of replacement for conventional electrode cables used in electrode connections. It is not overemphasized that configuration methods are not limited to methods illustrated in FIGS. 5 and 6, and those disclosed in the present invention are only served as examples to demonstrate their feasibility in real embodiment.

Based on above-described distributive configuration, an alternative of physiological monitoring apparatus used to measure single physiological parameter can be further simplified to gain additional benefits of efficient configuration, size reduction and power saving if some sharable elements in the node are distributed to other node. For example, some nodes are composed by amplifiers and A/D converters only, rather than those sharable elements, such as processor and RF transceiver.

Moreover, in another embodiment of the present invention, even a physiological monitoring apparatus employs only two electrodes, it also can be broken into plural distributive nodes that attach to skin surface via associated adhesive patches and connect to one another based on a configuration of node interconnections such as examples of FIGS. 5 and 6; that is, all the elements like processors, A/D converters, amplifiers and battery set in a node can be distributed to plural nodes so as to obtain even more compact nodes. As demonstrated in FIG. 8, all the circuits in a node of physiological monitoring apparatus are distributed to nodes 10″ and 10″′. For example, amplifier circuit (FIG. 8A) or battery set can be independently allocated in a node, or the amplifier circuit along with A/D converter are formed a node. It is noticed that quantity of nodes is not limited to two as described here and configuration of the circuits can be under any type of arrangement depending on practical considerations in real design, for example, both nodes may comprise a processor as shown in FIG. 8B.

Since one node is accompanied with one adhesive patch 20, attachment of the node to skin surface will not be an issue in above-described example. When multiple electrodes are used in physiological monitoring, for example, at least two electrodes required in ECG measurement and more than two required in ⅗-lead and 12-lead ECG, one may, for sure, simply adopts conventional electrode patch with snap fastener 811 (FIG. 8) as the adhesive patches for supporting the node, no matter the number of the nodes is fewer, equal or more than that of the electrode patch. In case, as more nodes appear in a configuration than electrodes do, non-electrode patches are adopted for extra nodes. Thereof, no restriction upon quantities of nodes and adhesive patches is imposed on any configuration of the present invention, and the result becomes quite obvious: the loading of the monitoring apparatus is distributed to plural adhesive electrode patches with extraordinary compact node thereon such that user even can't feel the existence of nodes.

In an embodiment, as multiple electrodes adopted, every electrode patch may need one amplifier for preventing signal attenuation so that if each amplifier is included in the associated node on the electrode patch, the node's size, which could be quite a burden if number of electrodes increases and all amplifiers are packed together inside a node, can be significantly reduced. While the method disclosed in the present invention can uniformly distribute loading of node (the ensemble of nodes) to every adhesive patches, and thus user could feel only patches, but not node.

In a preferred embodiment, the retrieved signals will be digitized first to prevent signal attenuation and reduce noise; whence more processors, A/D converters and amplifiers are required in such a case. Thereof, plural nodes may include plural processors, A/D converters and amplifiers; and, as described above, since not each type of circuit has to be included in each node, some circuits could be shared among several nodes for processing signals so as to reduce the size of nodes. Besides, another advantage of signal digitization in this case is the reduction of wiring complexity, which is, in one manner, caused by prevention of induced noises in wiring network.

Therefore, the above-described embodiments are only used to exemplify the flexibility of distributive configuration of circuits based on practical implementation; and the practice of present invention is not limited to these embodiments.

At this point, the physiological monitoring apparatus according to the present invention capable of measuring multiple physiological parameters, extending system based on designated configuration of circuits and adroitly attaching to user's skin surface has been posed. The user may implement versatile combinations of practice by way of above-described embodiments disclosed in the present invention. The following discloses detailed embodiments for implementing practical physiological monitoring apparatus.

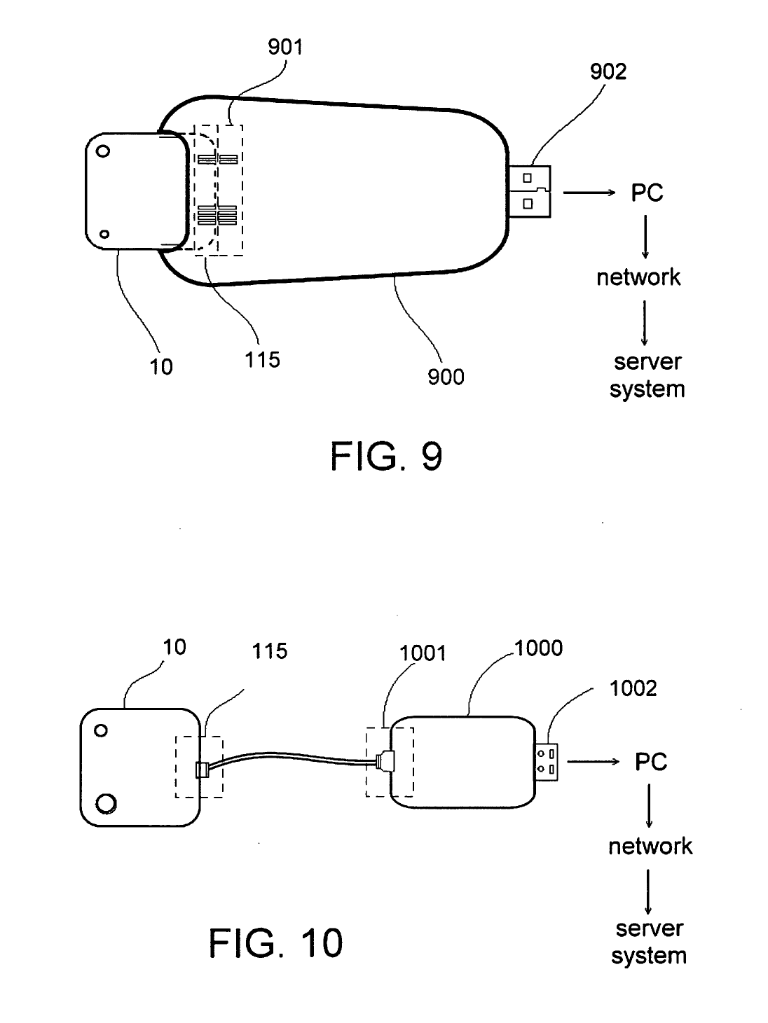

Referring to FIGS. 9 and 10, the present invention discloses a physiological monitoring system comprising at least an above-described physiological monitoring apparatus (including the node 10 and the adhesive patch for attachment to skin surface) and a RF transceiver 900 or 1000, wherein, as described above, a RF module and a connecting port 115 used for communication and power transmission are also included in the physiological monitoring apparatus. The RF transceiver 900, 1000, in addition to a RF module therein for wireless transmission/reception, includes a connecting port 901, 1001 (used for communication and power transmission) for connecting to the connecting port 115 on the node 10 and processing a handshaking therebetween. Moreover, the RF transceiver 900, 1000 are connected to a computer device via a communication port 902, 1002, for example, a USB port as illustrated in the figures, whence through a corresponding software on the computer device, the incoming physiological signals and information from the node 10 can be processed, stored and displayed on the computer device. Furthermore, through a communication between the RF modules of the node and the RF transceiver 900, 1000, users may, hereby, inspect and monitor physiological status at any moment and, through the settings of corresponding software on the computer device, the computer device may react to the retrieved physiological signals by way of voice, beep and graphics such as to provide guidance and/or warning to users for instant notification and avoid loss of timing due to carelessness. Besides, the apparatus may also link to a server system via a network connected to the computer device for performing advanced processes, for example, data update and integration, and, for sure, the server system may also feedback process results to the computer device for further utilization.

In addition, as the node is powered by a rechargeable battery, the function of power transmission included in both the connecting port 115 and the connecting port 901, 1001 is employed, whence performing charging process of the node upon their connection. That is, when electric power is in demand, the node can be charged simply through the same connecting operation between the RF transceiver and the node for handshaking, without further adapting extra charging device.

Therefore, in such an embodiment, on one hand, the RF transceiver can communicate with the physiological monitoring apparatus through the RF modules, that is, the RF transceiver can wirelessly receive signals from the physiological monitoring apparatus, and on the other hand, they can also exchange data and perform charging process through the connection of the connecting ports.

The connection between the connecting ports, for examples, may be accomplished through at least three methods. First of all, as indicated in FIG. 9, the node may connect to the wireless receiver by way of lock-and-key design upon their mechanical structure. The second example as indicated in FIGS. 7A and 10 demonstrates their connection through a connector with a wire. While FIG. 7B demonstrates another example that the connect-through-connector serves as a bridge between the node and the RF transceiver. Again, the methods disclosed here are to exemplify their implementation feasibility, and any embodiment will not be limited to them. Besides, whatever methods are used for connection, the difference between these methods is at their mechanical structures, rather than communication content and method between the node and the RF transceiver. Hence, it will not be specifically indicated this point in the following description.

Apart from above description, a mutual transmission between the connecting ports of the node and of the RF transceiver also includes handshaking, which entails ID authentication, hardware setting and signal transmission. Therefore, before monitoring in progress, the connection, either plug-in or connect-through-connector, between the node and the RF transceiver may easily initiate a matching process like channel designation and mutual identification. Once they are separated, the RF transceiver can determine which signals to be received based on prior matching, that is, signals from un-matched device will be ignored. Such a design renders the RF transceiver not to spend time on determination of which signals to be or not to be received, and thereof, to save power.

Furthermore, the RF transceiver can be implemented to communicate with plural physiological monitoring apparatuses, and for accomplishing this, it merely needs to connect plural nodes to the RF transceiver respectively to complete respective matching process, through which the RF transceiver can clearly identify what received signals originate.

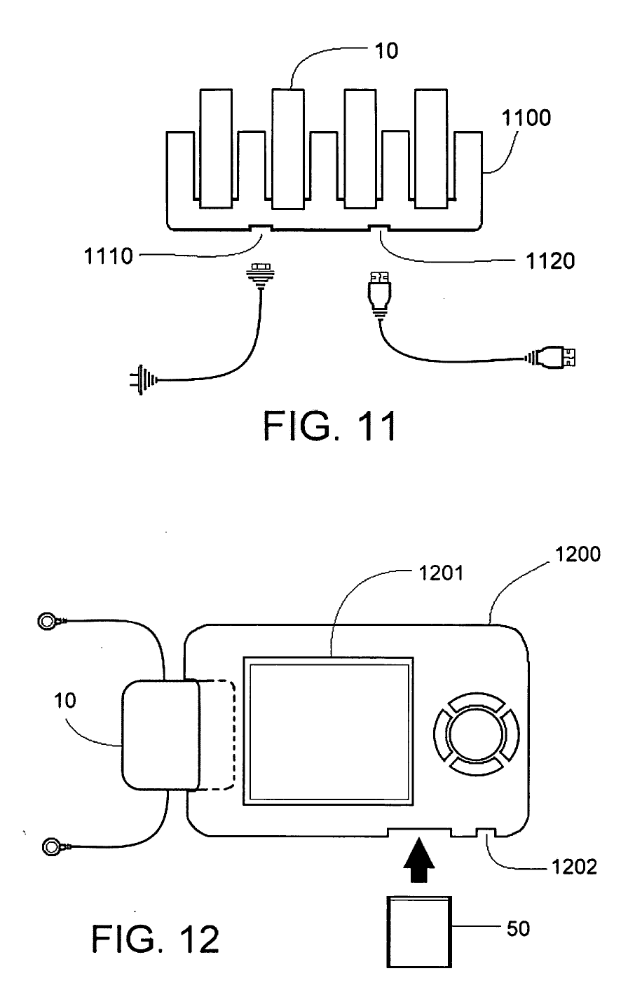

However, as a condition of plural physiological monitoring apparatuses disclosed in the present invention, no matter, for examples, plural patients in hospital or plural monitoring apparatuses applied to a user, sharing a common RF transceiver is employed, an alternative structure of RF transceiver 1100 that can receive multiple nodes 10 of plural physiological apparatuses is illustrated in FIG. 11 and disclosed here.

Thereof, the RF transceiver 1100 may, through a charging interface 1110, simply serve as a charging device for plural physiological apparatuses, or, through a communication port 1120, connect to a computer device to perform data exchange and charging for plural monitoring apparatuses simultaneously. In short, the user may plug a node 10 into a RF transceiver 1100 and proceed data exchange and charging process simultaneously when that node 10 is in idle, while unplug it to continue monitoring process without further operations about hardware identification and channel designation since these operations have been done as it is in the process of charging. It is, for sure, the embodiments could be any format following above-described scenario, and illustration in FIG. 11 is merely exemplified.

In another embodiment, the interface for charging process can be replaced by the communication port on the RF transceiver, rather than above-described charging interface. That is, the charging process is powered by a computer or any powered device with a communication port and could be easily accomplished through available devices without charging interface. The communication port disclosed here, depending on practical considerations, can be, but not limited to, USB, 1394 UART, SPI or any communication port with cable.

According to an advanced embodiment of present invention, the RF transceiver also can be implemented in a portable wireless operation device 1200, as shown in FIG. 12, to form an embodiment of alternative physiological monitoring system. The node in such a system also includes a RF module, a connecting port for communication and power transmission, and the adhesive patch is also employed to join the node 10 and attach to skin surface for support. Besides, the portable wireless operation device 1200, like above-described RF transceiver, comprises a RF module and a connecting port for communication and power transmission that may be linked to the connecting port on the node.

In one way, the portable wireless operation device wirelessly communicates with the physiological monitoring apparatus through the RF module; that is, the portable wireless operation device can wirelessly retrieve signals from the physiological monitoring apparatus. In another way, they accomplish data exchange through the connection between the connecting ports.

Similarly, the methods for connecting the two connecting ports can be, thereof, at least of three types, including plug-in (FIG. 12) and connect-through-connector (FIGS. 7A and 7B). It will not be further addressed here owing to prior disclosure.

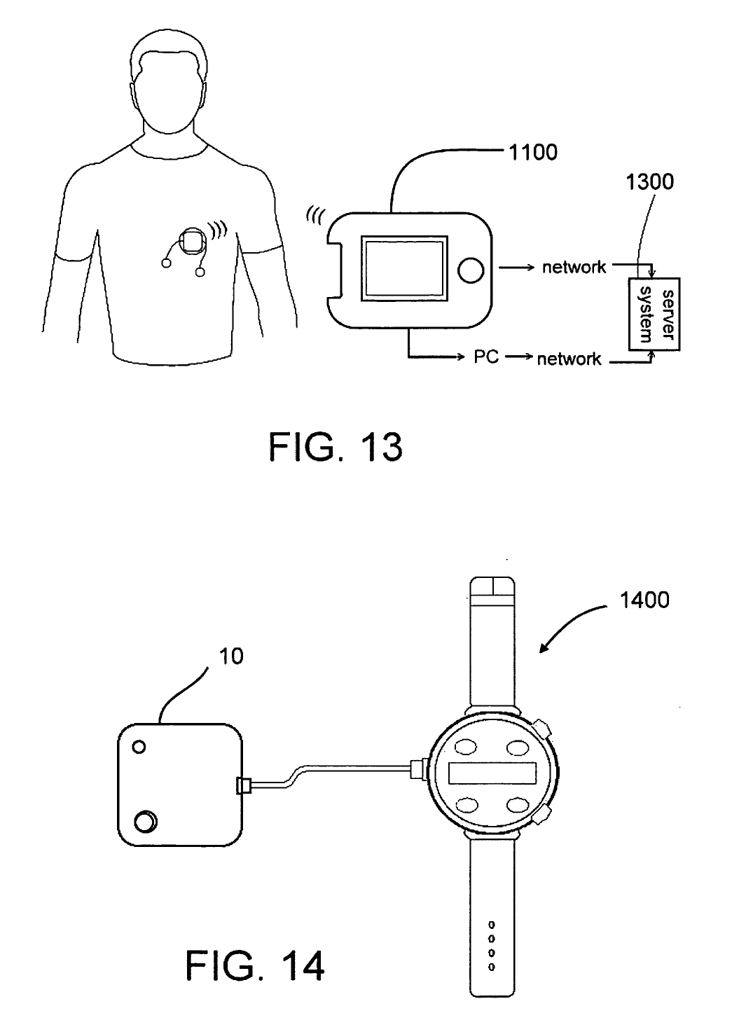

Similarly, apart from above description about connection between connecting ports, mutual transmission between them also includes handshaking, which entails ID authentication, hardware setting and signal transmission. The portable wireless operation device 1200 can, thereof, communicate with plural physiological monitoring apparatuses simultaneously and display physiological status and variation on its accompanied display device 1201. It will be appreciated that such a design may benefit users to learn their physiological status and variation in real-time. Since healthcare workers used to watch several patients simultaneously, the disclosed portable wireless operation device in the present invention may help them acquiring physiological status for each patient instantly, as shown in FIG. 13.

Moreover, the portable wireless operation device is further devised for reacting to retrieved physiological signals by way of voice, beep and graphics such as to provide guidance and/or warning to users for instant notification and avoid delay of medical aids. Since the portable wireless operation device is capable of network connection, it may link to a sever system 1300 for data exchange without other computer devices. Thereof, a central monitoring system on the server system may send warning calls and notify medical staffs for instant response to avoid delay of medical aids.

The portable wireless operation device 1200 may employ a communication port 1202, including, but not limiting to, USB, 1394, UART, SPI or any communication port with cable, to connect to a computer device. It may, like above-described RF transceiver, employ the computer to perform operations of display, warning and network connection. The details are not repeated here.

Furthermore, when the battery included in the node, like that of the RF transceiver described above, is rechargeable, the charging process can be accomplished by connecting the connecting ports of the node and of the portable wireless operation device through the function of power transmission thereof. However, unlike the RF transceiver described above, except charging the node through a portable wireless operation device connected-computer, the portable wireless operation device per se may also supply electric power to charge the node if rechargeable battery and external power supply are equipped in the portable wireless operation device.

It is especially noticed that additional external memory set 50, in addition the equipped memory set in the portable wireless operation device, is also available to meet user's demands on more and lengthy recordings. The external memory set disclosed here can be a conventional memory card or stick, and even a portable hard drive.

Another noticeable point is the styles of portable wireless operation device disclosed in the present invention can be, but not limited to, a watch 1400 (FIG. 14), neckpiece, or any other portable styles, such as cell phone and PDA, although the device illustrated in FIGS. 12 and 13 is hand-held.

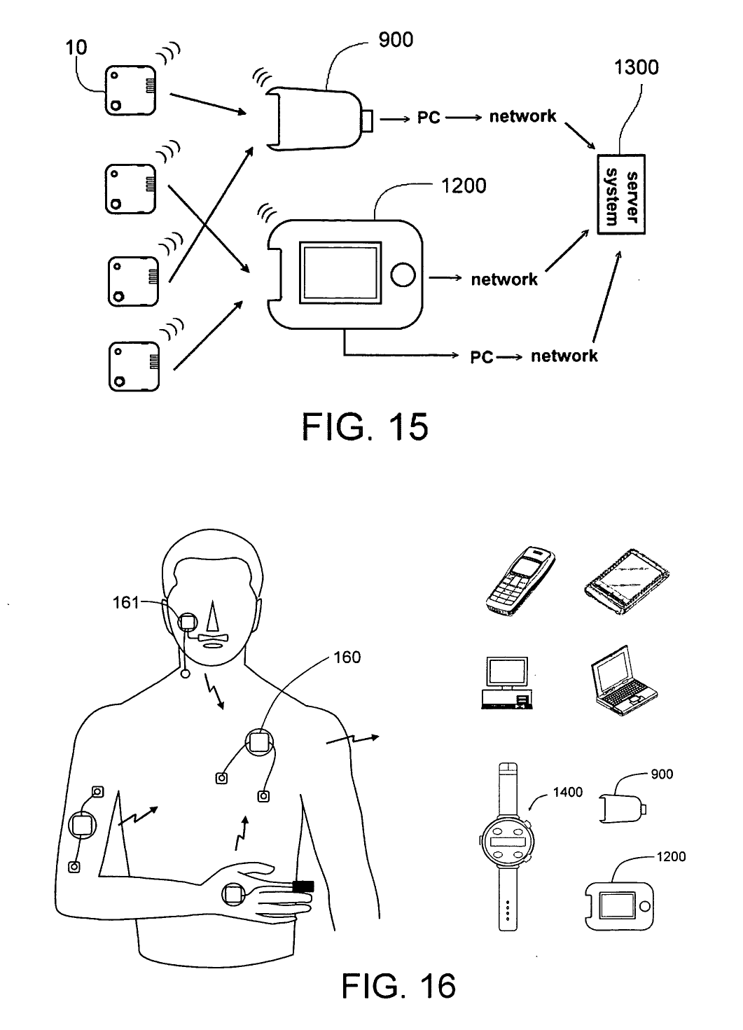

The present invention also relates to a type of physiological monitoring network, as illustrated in FIG. 15, that comprises at least a physiological monitoring apparatus, at least a RF transceiver 900 and/or at least a portable wireless operation device 1200 and a server system 1300. Likewise, the disclosed physiological monitoring apparatus, together with the RF transceiver and/or the portable wireless operation device, can perform handshaking, which entails ID authentication, hardware setting and signal transmission, through the connecting ports, as well as establish a matching between respective physiological monitoring apparatus and the RF transceivers or the portable wireless operation devices for avoiding confusion among various match connections. Then, by way of computers and network, physiological signals can be instantly transmitted to the server system for performing operations of monitoring/analyzing/processing/storing/informing related personnel. Thereof, it is easily and adroitly to achieve real-time physiological monitoring network in a region, such as a building or campus, by way of the physiological monitoring network disclosed in the present invention. Moreover, such a physiological monitoring network may also convey analyzed results performed by the server system back to the portable wireless operation device 1100 and/or the computer devices, which usually demand much higher loading of computations or database operations and is hardly processed by a portable wireless operation device and/or computer device.

The following details another embodiment of physiological monitoring network.

To apply the concepts and principles of the present invention to physiological monitoring, in addition to the embodiments exemplified in the above description regarding a communication mode allowing of wireless transmission/reception between disclosed RF transceiver and/or disclosed portable wireless operation device and the node(s), another communication mode between nodes is also disclosed in the present invention, shown in FIG. 16, where one of the nodes serves as a master node 1600, while others serve as slave nodes, in the configuration of multiple physiological monitoring apparatuses. Thereof, the physiological signals retrieved from slave nodes are transmitted to the master node through their RF modules, and then, collected physiological signals are transmitted to an external device, such as a desktop/laptop personal computer, PDA, cell phone and any other RF device, for example, the above-described RF transceiver and the portable wireless operation device disclosed in the present invention.

In another embodiment, plural slave nodes may link together through cable connectors, and one of them communicates with the master node wirelessly.

In an advanced embodiment, the physiological monitoring apparatus used in the multiple physiological monitoring is not restricted to retrieve one type of physiological signals; instead, two or more types of physiological signals are allowed to be retrieved by one physiological monitoring apparatus. For example, the physiological monitoring apparatus 1601 shown in FIG. 16 retrieves both breathing and snoring signals simultaneously.

In such a master-slave communication mode, the matching connection for data exchange with external devices is valid for the master node, rather than slave nodes, since all slave nodes perform data exchange with external devices only through the master node. Although slave nodes may perform matching connections with external devices, they are supposed to do so only for establishing the matching with the master node by the operation interface or software on an external device.

In addition to wireless communication described above, the master node is able to synchronize actions of other slave nodes, including controls, settings, initiation, termination and data exchange, in the mode of master-slave communication. Users may thus easily control slave nodes through controlling the master node, in which additional operation interface and display device may also be incorporated to help operations of synchronization. Since master node is responsible to communicate with external devices, remote control is feasible, and thus, one may send commands to the master node from an external device, then the slave nodes attached to user's skin surface may execute remote commands relayed from the master node.

Apparently, the above-described method is especially useful in hospital and/or homecare monitoring of physiology. In an embodiment of hospital application, the disclosed external device can be a computer hosted in a sick ward and/or nurse station, the server system in a hospital, or a portable wireless operation device disclosed in the present invention carried by physicians and nurses, and can be used to control plural master nodes attached to plural patients to achieve real-time monitoring. It is especially beneficial as long-term physiological monitoring is required.

In addition, as in an embodiment of homecare monitoring of physiology, the external device can be a personal computer, which is directly connected to the monitoring apparatus or is indirectly connected thereto through the disclosed RF transceiver, a PDA, a cell phone, or the portable wireless operation device disclosed in the present invention. Thereby, the user can easily operate multiple physiological monitoring apparatuses through the external device. It is especially benefit in baby and old people care.

In embodiment of present invention, the retrieved physiological signals can be saved in memory set of the master node for permanent storage or temporary use as a buffer of data transmission (The details of memory set has been described above and not repeated here). Users may utilize the memory set in either master node, external device (such as personal computer) or portable wireless operation device disclosed in the present invention to perform long-term monitoring of physiology at home and bring the master node or the associated memory card where data were recorded to hospital for further medical consultation. By way of such utilization of the present invention enables patients to perform long-term, periodic and real-time monitoring of physiology, and, thereof, unlashes the reins to limited medical resource in hospital.

The embodiment illustrated in FIG. 16 still exists other variety as exemplified below.

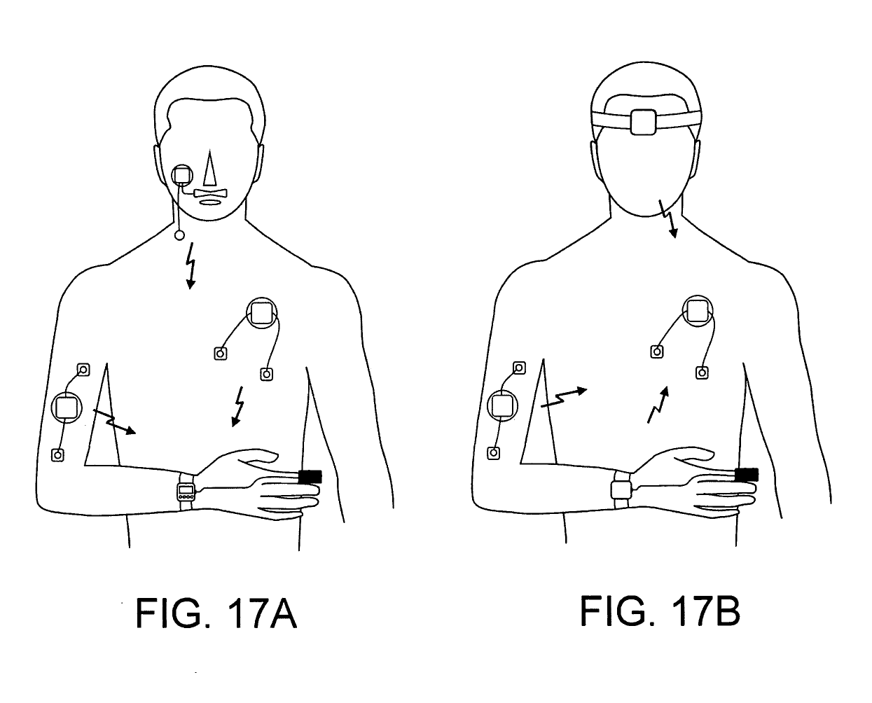

For example, the above-described adhesive patches for nodes attaching to skin surface can be selectively replaced by other attaching elements, such as a belt, at certain measuring sites (such as wrist, arm and head) to ease production and application (FIGS. 17A and 17B).

It is noticeable that utilization of non-adhesive patch methods to attach nodes to body surface is not restricted to the master node, but depends on geometry of measuring site. For instance, the node is allocated to wrist as a watch style when pulse oximeter probe is placed on fingertip. Another example of electromyography (EMG) and position measurement of legs may utilize a belt to hold node and associated elements. Or, a belt holding EEG apparatus may be applied to head.

To sum up, a portable physiological monitoring apparatus featured as extraordinary compact and light weight is accomplished by accompanied with designs of compact node and associated adhesive patches in the present invention. Especially, a distributive configuration of nodes and associated functional circuitry enables multiple electrode/sensor measurements without increasing size and complexity. Besides, due to the physiological monitoring apparatus of the present invention applied to a variety of physiological measuring devices, such as ECG, EMG and oxygen desaturation, testers, when multiple monitoring of physiological parameters are required (such as polysomnography), no longer need to carry a clumsy and wire-tangling system as described in prior arts for monitoring of physiological parameters; instead, the present invention provides an ensemble of compact and adroit nodes attaching to measuring sites to perform multiple physiological monitoring. Then, through a signal synchronization among nodes, for example, physiological signals are integrated and transmitted through either a RF transceiver disclosed in the present invention or the present-inventive portable wireless operation device, or signals from multiple nodes are integrated and transmitted through the master-slave communication mode between nodes, the effect of multiple monitoring of physiological parameters the same as the conventional PSG monitoring can be achieved. The present invention, not only reduces problematic wire-tangling and system size, but also removes the mobile deficiency in prior arts, such that long-term multiple monitoring of physiological parameters can be realized at home.

Apart from wireless communication with the disclosed RF transceiver/portable wireless operation device for signal transmission/reception, the nodes in above-described physiological monitoring system proceed to a temporary connection (in form of plug-in and connect-through-connector) with them to initiate handshaking including ID authentication and set up master-slave matching connections among these nodes, thus establishing a stable wireless transmission.

Moreover, the disclosed RF transceiver can display physiological status and connect to network and server system by ways of a communication port connecting to a computer device. The portable wireless operation device can even perform operations of connecting to a network directly or through a computer device, and to a server system. Besides, as rechargeable battery is adopted in the disclosed physiological monitoring apparatus, the RF transceiver/the portable wireless operation device can operate as a charging device. All these designs remove deficiencies found in applications of hospital and homecare.

And it is most noticeable that conventional electrodes are fully applied to the disclosed physiological monitoring apparatus in the present invention; whence benefit the users who already own conventional physiological monitoring apparatus. For example, users in hospital can adopt conventional electrode accessories, accompanied with the node disclosed in the present invention featured as compact and cost-efficient, to perform physiological monitoring through operations on a computer device or portable wireless operation device. If users further apply physiological monitoring network disclosed in the present invention to hospital, campus and/or community homecare, it is foreseeable that the limited medical resource will be most efficiently utilized.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

What is claimed is:

1. A physiological monitoring apparatus, comprising:
at least a node; and
at least a patch for attaching to a skin surface of a user and for supporting the node on the skin surface through joining therewith, wherein
the node comprises:
at least a signal I/O port for externally connecting to at least a sensor or electrode through a connecting wire so as to acquire a physiological signal; and
a RF module for transmitting and receiving signal.

2. An apparatus as claimed in claim 1, wherein the apparatus is performed to measure one single type of physiological signals or multiple types of physiological signals, the patch is performed to be an electrode for sensing physiological signals, a reference and/or ground electrode or a temperature sensor, the patch is jointed with the node by snap fastener, pocket pouch, adhesive binder, strap fastener or any other join methods, the connecting wire is connected with the signal I/O port through plug-in, connect-through-connector or direct connection, and the RF module is Bluetooth, 802.11x, GPS, IrDA or any other wireless devices.

3. An apparatus as claimed in claim 1, wherein the node further comprises a power switch, a display device for display and indication, and an operation interface having a push button for time and event markings by user.

4. An apparatus as claimed in claim 1, wherein the node further comprises a processor, an analog signal processing circuitry, and a battery set which is rechargeable, and the node further comprises a power input and a power output for power relay between plural nodes.

5. An apparatus as claimed in claim 1, wherein the number of the node is performed to be plurality.

6. An apparatus as claimed in claim 5, wherein the circuits and structures consisting of the physiological monitoring apparatus are distributed in plural nodes, and the node further comprises an extension port for connecting to another extension port of another node through connecting wire so as to communicate therebetween.

7. An apparatus as claimed in claim 5, wherein the physiological monitoring apparatus is performed to measure one single type of physiological signals or multiple types of physiological signals.

8. An apparatus as claimed in claim 5, further comprising at least an attaching element for combining with at least one of the nodes so as to attach the at least one node on the skin surface

9. An apparatus as claimed in claim 5, wherein one of the nodes is performed to be a master node, and the master node is capable of synchronizing, setting, and integrating other nodes and capable of outputting the received signals and the signals acquired thereby.

10. An apparatus as claimed in claim 9, wherein other nodes are performed to wirelessly transmit signals to the master node, and/or wherein the other nodes are interconnected in a wired manner and at least one of the other nodes has a RF module for wirelessly communicating with the master node.

11. An apparatus as claimed in claim 1, wherein the RF module is performed to execute a real time data transmission

12. An apparatus as claimed in claim 1, wherein the node further comprises a memory set for data storage and the obtained data is stored in the memory set at first and then transmitted by the RF module.

13. An apparatus as claimed in claim 1, wherein the node further comprises a connecting port used for communication and power transmission with another device so as to accomplish a data transmission therebetween, and the node further comprises a memory set for data storage, the obtained data being stored in the memory set at first and then transmitted by the connecting port.

14. An apparatus as claimed in claim 1, wherein the number of the signal I/O port is performed to be plurality so as to connect to plural electrodes and/or sensors, and wherein the physiological signal obtained by the electrode and/or sensor is at least one of the group consisting of: an ECG signal, a EEG signal, a EOG signal, a EMG signal, a snoring signal, a respiratory signal, a thorax/abdominal breathing effort signal, a limb movement signal, a torso movement signal, a head movement signal and a signal indicating blood oxygen level.

15. A physiological monitoring apparatus, comprising:
at least a node; and
at least a patch for attaching to a skin surface of a user and for supporting the node on the skin surface through joining therewith, wherein
the node comprises:
at least a signal I/O port for externally connecting to a sensor or an electrode through a connecting wire so as to acquire a physiological signal; and
a connecting port used for communication and power transmission with an external device.

16. An apparatus as claimed in claim 15, wherein the external device is a wireless device having a connecting port used for communication and power transmission for combining with the connecting port of the node.

17. An apparatus as claimed in claim 16, wherein the physiological monitoring apparatus and the wireless device are combined through combining the connecting port of the node with the connecting port of the wireless device so as to achieve a handshaking therebetween, including ID authentication, hardware settings and signal transmission.

18. An apparatus as claimed in claim 16, wherein the wireless device further comprises a RF module and the physiological monitoring apparatus further comprises a RF module so that a wireless communication therebetween is achieved.

19. An apparatus as claimed in claim 16, wherein the wireless device is performed to electrically connect with plural physiological monitoring apparatuses.

20. An apparatus as claimed in claim 16, wherein the connecting port of the wireless device is a wired communication interface which is a USB, a RS-232, a 1394, a UART, or any other wired communication ports, and wherein the connection between the connecting port of the wireless device and the connecting port of the node is achieved by a lock-and-key design, in which the connecting port of the wireless device is performed to have a socket matching with an outer profile of the node, a transmission wire, or a connector.

21. An apparatus as claimed in claim 16, wherein the wireless device further comprises a display device for displaying signals from the at least a physiological monitoring apparatus and for guiding and/or warning the user via character, audio and/or graph corresponding to the signals, or the wireless device is further connected to a computer device for display the signals from the at least a physiological monitoring apparatus and guide and/or warn the user via character, audio and/or graph corresponding to the signals, and wherein the wireless device is performed to have a memory set, or to externally connect to a memory set, which is one of a memory card/stick, a portable hard drive and a flash disc.

22. An apparatus as claimed in claim 16, wherein the wireless device is performed to directly connect to a network and further to a server system, or the wireless device further comprises a communication port for connecting to a computer device to connect to a network and further to a server system, the communication port being a USB, a 1394, a UART, a SPI or any other wired communication ports.

23. An apparatus as claimed in claim 16, wherein the wireless device is one of a RF receiver and a portable wireless operation device, and the portable wireless operation device is one selected from a group consisting of: a handheld device, a watch-type device, a neck-hanged device, and other portable devices.

24. A physiological monitoring network, comprising:
at least a physiological monitoring apparatus, comprising:
at least a node having a connecting port used for communication and power transmission and a wireless transmission module; and
at least a patch for attaching to a skin surface of a user and for supporting the node on the skin surface through joining therewith;
at least a wireless device having a connecting port for communication and power transmission with the connecting port of the node; and
a server system for real time monitoring, analyzing, processing, storing and/or informing related personnel, wherein
the communication between the physiological monitoring apparatus and the wireless device comprises a handshaking; and
a communication between the wireless device and the server system is achieved in a wired or wireless manner.
